(19) United States
(12) Patent Application Publication
(10) Pub. No.: US 2025/0018066 A1
(43) Pub. Date: Jan. 16, 2025

(54) Ophthalmic dye composition

(71) Applicant: Vitreq B.V., Vierpolders (NL)

(72) Inventors: Detlef Gabel, Bremen (DE); Andreas Matthias Mohr, Bremen (DE); Frank Ruseler, Schiedam (NL)

(73) Assignee: Vitreq B.V., Vierpolders (NL)

(21) Appl. No.: 18/772,309

(22) Filed: Jul. 15, 2024

Related U.S. Application Data
(62) Division of application No. 16/093,477, filed on Oct. 12, 2018, now abandoned.

(30) Foreign Application Priority Data
Apr. 22, 2016 (EP) 16166602.9

(51) Int. Cl. A61K 49/00; A61F 9/00; A61F 9/007

(52) U.S. Cl. CPC A61K 49/006; A61F 9/0008; A61F 9/007; A61K 49/0023; A61K 49/003

(57) ABSTRACT

The present invention relates to the field of medicine, specifically to ophthalmic surgery, more specifically to an improved staining composition for ophthalmic surgery with low toxicity and increased staining efficiency.

### FIELD OF THE INVENTION

The present invention relates to the field of medicine, specifically to ophthalmic surgery, more specifically to an improved staining composition for ophthalmic surgery with low toxicity and increased staining efficiency.

### BACKGROUND OF THE INVENTION

Dyes are used frequently in ophthalomoligical procedures to stain tissues of interest. For anterior segment surgery, trypan blue (TB) (CAS number 72-57-1) is the most commonly used dye for anterior capsule, deep lamellar keratoplasty dissection, keratoplasty, Descemet's membranes, cataract surgery, conjunctival surgery, and anterior vitreous surgery [1].

In vitreo-retinal surgery, two different structures need to be removed in certain pathological situations in order to restore vision: the epiretinal membrane (ERM) (an abnormal structure generated e.g. when the patient suffers from chronic diabetes) and the internal limiting membrane (ILM), which separates the vitreous from the retina, and is firmly attached to the latter.

For surgery, the vitreous is removed, and the eye is filled either with gas or with a salt solution in preparation for removal of the ERM and the ILM from the retina. The state of the art is to visualize the structures by staining them with dyes and then to remove them.

The first dye used in ILM staining was indocyanine green (ICG) (CAS number 3599-32-4). Its spectral properties with an absorption maximum outside the range visible to the eye has limited its usefulness [2]. Staining of the ERM is often done with TB. TB does, however, not stain the ILM strongly [2, 3]. Therefore Brilliant Blue G (BBG) (CAS number 6104-58-1) has been added to TB, in order to visualize also the ILM [4]; it is also used alone. The staining which can be achieved with TB and with BBG, when compared, have been reported as equal, and inferior to that of ICG [5]. Recently, Brilliant Blue G (BBG) (CAS number 6104-58-1) has been combined with Bromophenyl Blue (BP) (CAS number 115-39-9). Acid Violet 17 (CAS number 4129-84-4), which was also recently introduced, has been reported as toxic. All of these dyes and dye combinations thus appear to have drawbacks and risks involved. Concentrations of the dyes must be high enough to allow appropriate staining; a reduction in the concentration will usually lead to staining reduced to so low levels that they are of little value for the surgeon. As an alternative to a dye, or in combination with a dye, the highly insoluble Triamcinolone Acetonide (TA) has been used for aiding the surgeon in locating the ILM [3]. The opaque crystalline flakes of this colorless substance sediment to the membrane, and areas which have been removed can be recognized by the absence of the white flakes. When the eye is filled with salt solution, the dye solution is injected into the salt solution and should then sink, by gravity, onto the retina. Different additives are used to increase density and viscosity. One additive is a polymer, such as polyethylene glycol (PEG). PEG has the disadvantage that it reduces the staining and thereby the visibility of the ILM. Other additives are sugars or sugar alcohols. These have the disadvantage that they contribute to the osmotic pressure and thus to cell death if not balanced by a reduction in other osmotically active substances. Therefore, their maximum concentration, and as a consequence the density of the solution which can be applied safely, is limited. More importantly, the staining effect of the dye can be reduced severely by some of these additives, especially polymeric additives.

A good additive should therefore not reduce the staining. Ideally, an additive for increasing the density should rather enhance the effect of the staining. Finally, any solution used to stain the ILM and the ERM should exhibit low toxicity, in order to not to endanger the results of the surgery. Accordingly, there is a need for an improved staining composition with low toxicity and increased staining efficiency.

### SUMMARY OF THE INVENTION

In an aspect, the present invention provides for a composition suitable for staining an ophthalmic structure comprising Trypan Blue or a pharmaceutically acceptable salt or hydrate thereof and further comprising a dye selected from the group consisting of Methyl Blue, Methylene Blue, Janus Green, Methyl Green, Crystal Violet, Methyl Violet, Ethyl Violet, Evans Blue and their respective pharmaceutically acceptable salts and hydrates thereof.

The invention further provides for such composition, wherein,
Methyl Blue, if present, has a concentration within the range of about 0.001% (w/v) to about 0.4% (w/v), preferably within the range of about 0.0025% (w/v) to about 0.1% (w/v),
Methylene Blue, if present, has a concentration within the range of about 0.001% (w/v) to about 0.1% (w/v), preferably within the range of about 0.01% (w/v) to about 0.1% (w/v),
Janus Green, if present, has a concentration within the range of about 0.001% (w/v) to about 0.1% (w/v), preferably within the range of about 0.005% (w/v) to about 0.01% (w/v),
Methyl Green, if present, has a concentration within the range of about 0.001% (w/v) to about 0.1% (w/v), preferably within the range of about 0.01% (w/v) to about 0.05% (w/v),
Crystal Violet, if present, has a concentration within the range of about 0.001% (w/v) to about 0.1% (w/v), preferably within the range of about 0.01% (w/v) to about 0.05% (w/v),
Methyl Violet, if present, has a concentration within the range of about 0.001% (w/v) to about 0.1% (w/v), preferably within the range of about 0.01% (w/v) to about 0.05% (w/v),
Ethyl Violet, if present, has a concentration within the range of about 0.001% (w/v) to about 0.1% (w/v), preferably within the range of about 0.01% (w/v) to about 0.05% (w/v),
Evans Blue, if present, has a concentration within the range of about 0.001% (w/v) to about 0.4% (w/v), preferably within the range of about 0.0025% (w/v) to about 0.1% (w/v),
Indocyanine Green, if present, has a concentration within the range of about 0.001% (w/v) to about 0.4% (w/v), preferably within the range of about 0.0025% (w/v) to about 0.1% (w/v), and,
Trypan Blue has a concentration within the range of about 0.001% (w/v) to about 0.25% (w/v), preferably within the range of about 0.01% (w/v) to about 0.15% (w/v).

The invention further provides for a composition suitable for staining an ophthalmic structure comprising at least a dye and further comprising diglycerol and/or triglycerol as an agent to increase the density and viscosity of the composition.

The invention further provides for a composition according to the invention, further comprising diglycerol and/or triglycerol as an agent to increase the density and viscosity of the composition.

The invention further provides for a composition according to the invention, wherein the total concentration of the diglycerol and/or triglycerol is within the range of about 0.1% to about 25% (v/v), preferably within the range of about 2% to about 20% (v/v), more preferably within the range of about 3% to about 6% (v/v).

The invention further provides for a composition suitable for staining an ophthalmic structure comprising at least a dye and further comprising a Gadolinium complex to enhance the density and to enhance staining of the ophthalmic structure.

The invention further provides for a composition according to the invention, further comprising a Gadolinium complex to enhance the density and to enhance staining of the ophthalmic structure.

The invention further provides for a composition according to the invention, wherein the concentration of the Gadolinium complex is within the range of about 0.01 M to about 0.1.M, preferably within the range of about 0.01M to about 0.05 M, more preferably in the range of about 0.02M to about 0.04M.

The invention further provides for a kit of parts comprising a composition according to the invention, wherein the kit comprises the compounds within a single composition or comprises the compounds in multiple compositions.

The invention further provides for a composition according to the invention, or a kit of parts according to the invention for use as a medicament, preferably for use in a method of ophthalmic surgery.

The invention further provides for a composition for use as a medicament according to the invention, wherein the ophthalmic surgery comprises staining of an ophthalmic structure.

The invention further provides for a composition for use as a medicament according to the invention, wherein the ophthalmic structure is the internal limiting membrane (ILM) and/or the epiretinal membrane (ERM) and/or the anterior cavity.

### DETAILED DESCRIPTION OF THE INVENTION

Surprisingly, it has now been demonstrated that by using a combination of Trypan Blue (TB) with another dye, staining of the ILM can be greatly enhanced over that of each dye alone, and over the staining achieved with any of the prior art combinations of dyes. Combinations of TB with Methyl Green (MG) (CAS number depending on additives: ZnCl₂: 7114 Mar. 6; none: 14855-76-6), Crystal Violet (CV) (CAS number 548-62-9), Methyl Violet (MV) (CAS number 8004-87-3), Ethyl Violet (EV) CAS number 2390-59-2), Evans Blue (EB) (CAS number 314-13-6), Janus Green B (JG) (CAS number 2869-83-2), Methyl Blue (MeB) (CAS number 28983-56-4), and Methylene Blue (MB) (CAS number 61-73-4) were found to be excellent combinations to improve the staining of the ILM. They stain, in an unexpected manner, the ILM better than what can be achieved either with TB alone or with the secondary dye alone.

Accordingly, in a first aspect the present invention provides a composition suitable for staining an ophthalmic structure comprising Trypan Blue or a pharmaceutically acceptable salt or hydrate thereof and further comprising a dye selected from the group consisting of Methyl Blue, Methylene Blue, Janus Green, Methyl Green, Crystal Violet, Methyl Violet, Ethyl Violet, Evans Blue and their respective pharmaceutically acceptable salts and hydrates thereof. Such composition is herein referred to as a composition according to the invention. A preferred composition according to the invention comprises Trypan Blue and Methyl Blue, Trypan Blue and Methylene Blue or Trypan Blue and Janus Green. The dyes herein can be purchased since they are commercially available; they may also be manufactured using common knowledge of the person skilled in the art. The purity of the dyes is preferably as high as possible, preferably of pharmaceutical grade, although the dyes herein are not necessarily limited thereto. Preferably, the purity of a dye herein is at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or most preferably at least 99% pure. Examples of pharmaceutical acceptable salts include but are not limited to, salts formed with organic bases (such as glucosamine, galactosamine, mannosamine, meglumine, trimethylamine, choline, procaine, triethanolamine, diethanolamine, and ethanolamine), inorganic bases (such as ammonia, alkaline metals, and alkaline earth metals), organic acids (such as para-toluene sulfonic acid, methane sulfonic acid, formic acid, trifluoro acetic acid, and maleic acid), inorganic acids (such as hydrochloric acid, sulfuric acid, phosphoric acid and nitric acid), basis amino acids (such as lysine, arginine, histidine and ornithine), halogen ions (such as F and Cl ions) and intramolecular salts. The composition according to the invention can conveniently be used in ophthalmic manipulations on a subject such as ophthalmic surgery, preferably as an ophthalmic adjuvant. Examples of diseases and conditions of the eye that require ophthalmic manipulations such as ophthalmic surgery are, but are not limited to, vitreo-retinal disease such as macular hole, retinal detachment due to hymoyopathic macular hole, epiretinal membrane, proliferative diabetic retinopathy, diabetic macular edema, proliferative vitreoretinopathy, specific cataracts such as hypermature cataract, congenital cataract, and split thickness corneal transplantation. The subject is preferably a mammal, more preferably a human.

The composition according to the invention can be in any form, such as a mixture of solids and a liquid, preferably an aqueous liquid. All components may be present in a single composition or may be present in different compositions that are mixed before use; all (dye) components may be present in a solid mixture that is dissolved before use; for preparation it may conveniently be dissolved in intraocular cleaning solution, intraocular rinsing solution, physiological saline or a balanced salt solution. The composition according to the invention may comprise or may be mixed with a pharmaceutically acceptable excipient and/or carrier and/or a drug known to the persons skilled in the art. After preparation, the composition according to the invention may be sterilized, e.g. by filtration or autoclaving.

Preferably, the pH of the solution is within the range of pH 7.0 to pH 7.6, more preferably in the range of pH 7.1 to pH7.5, more preferably in the range of pH7.2 to pH7.5, more preferably in the range of pH7.3 to pH7.5 even more preferably the pH is physiological, i.e. neutral, i.e. about pH 7.4, most preferably the pH is 7.4.

Preferably, in a composition according to the invention:
Methyl Blue, if present, has a concentration within the range of about 0.001% (w/v) to about 0.4% (w/v), preferably within the range of about 0.0025% (w/v) to about 0.1% (w/v),
Methylene Blue, if present, has a concentration within the range of about 0.001% (w/v) to about 0.1% (w/v), preferably within the range of about 0.01% (w/v) to about 0.1% (w/v),
Janus Green, if present, has a concentration within the range of about 0.001% (w/v) to about 0.1% (w/v), preferably within the range of about 0.005% (w/v) to about 0.01% (w/v),
Methyl Green, if present, has a concentration within the range of about 0.001% (w/v) to about 0.1% (w/v), preferably within the range of about 0.01% (w/v) to about 0.05% (w/v),
Crystal Violet, if present, has a concentration within the range of about 0.001% (w/v) to about 0.1% (w/v), preferably within the range of about 0.01% (w/v) to about 0.05% (w/v),
Methyl Violet, if present, has a concentration within the range of about 0.001% (w/v) to about 0.1% (w/v), preferably within the range of about 0.01% (w/v) to about 0.05% (w/v),
Ethyl Violet, if present, has a concentration within the range of about 0.001% (w/v) to about 0.1% (w/v), preferably within the range of about 0.01% (w/v) to about 0.05% (w/v),
Evans Blue, if present, has a concentration within the range of about 0.001% (w/v) to about 0.4% (w/v), preferably within the range of about 0.0025% (w/v) to about 0.1% (w/v),
Indocyanine Green, if present, has a concentration within the range of about 0.001% (w/v) to about 0.4% (w/v), preferably within the range of about 0.0025% (w/v) to about 0.1% (w/v), and,
Trypan Blue has a concentration within the range of about 0.001% (w/v) to about 0.25% (w/v), preferably within the range of about 0.01% (w/v) to about 0.15% (w/v).

More preferably, in a composition according to the invention:
Methyl Blue, if present, has a concentration within the range of 0.001% (w/v) to 0.4% (w/v), preferably within the range of 0.0025% (w/v) to 0.1% (w/v),
Methylene Blue, if present, has a concentration within the range of 0.001% (w/v) to 0.1% (w/v), preferably within the range of 0.01% (w/v) to 0.1% (w/v),
Janus Green, if present, has a concentration within the range of 0.001% (w/v) to 0.1% (w/v), preferably within the range of 0.005% (w/v) to 0.01% (w/v),
Methyl Green, if present, has a concentration within the range of 0.001% (w/v) to 0.1% (w/v), preferably within the range of 0.01% (w/v) to 0.05% (w/v),
Crystal Violet, if present, has a concentration within the range of 0.001% (w/v) to 0.1% (w/v), preferably within the range of 0.01% (w/v) to 0.05% (w/v),
Methyl Violet, if present, has a concentration within the range of 0.001% (w/v) to 0.1% (w/v), preferably within the range of 0.01% (w/v) to 0.05% (w/v),
Ethyl Violet, if present, has a concentration within the range of 0.001% (w/v) to 0.1% (w/v), preferably within the range of 0.01% (w/v) to 0.05% (w/v),
Evans Blue, if present, has a concentration within the range of 0.001% (w/v) to 0.4% (w/v), preferably within the range of 0.0025% (w/v) to 0.1% (w/v),
Indocyanine Green, if present, has a concentration within the range of 0.001% (w/v) to 0.4% (w/v), preferably within the range of 0.0025% (w/v) to 0.1% (w/v), and,
Trypan Blue has a concentration within the range of 0.001% (w/v) to 0.25% (w/v), preferably within the range of 0.01% (w/v) to 0.15% (w/v).

Even more preferably, in a composition according to the invention:
Methyl Blue, if present, has a concentration within the range of about 0.025% (w/v) to about 0.1% (w/v),
Methylene Blue, if present, has a concentration within the range of about 0.01% (w/v) to about 0.1% (w/v),
Janus Green, if present, has a concentration within the range of about 0.005% (w/v) to about 0.01% (w/v),
Methyl Green, if present, has a concentration within the range of about 0.01% (w/v) to about 0.05% (w/v),
Crystal Violet, if present, has a concentration within the range of about 0.01% (w/v) to about 0.05% (w/v),
Methyl Violet, if present, has a concentration within the range of about 0.01% (w/v) to about 0.05% (w/v),
Ethyl Violet, if present, has a concentration within the range of about 0.01% (w/v) to about 0.05% (w/v),
Evans Blue, if present, has a concentration within the range of about 0.025% (w/v) to about 0.1% (w/v),
Indocyanine Green, if present, has a concentration within the range of about 0.025% (w/v) to about 0.1% (w/v), and,
Trypan Blue has a concentration within the range of about 0.01% (w/v) to about 0.25% (w/v).

Even more preferably, in a composition according to the invention:
Methyl Blue, if present, has a concentration within the range of 0.025% (w/v) to 0.1% (w/v),
Methylene Blue, if present, has a concentration within the range of 0.01% (w/v) to 0.1% (w/v),
Janus Green, if present, has a concentration within the range of 0.005% (w/v) to 0.01% (w/v),
Methyl Green, if present, has a concentration within the range of 0.01% (w/v) to 0.05% (w/v),
Crystal Violet, if present, has a concentration within the range of 0.01% (w/v) to 0.05% (w/v),
Methyl Violet, if present, has a concentration within the range of 0.01% (w/v) to 0.05% (w/v),
Ethyl Violet, if present, has a concentration within the range of 0.01% (w/v) to 0.05% (w/v),
Evans Blue, if present, has a concentration within the range of 0.025% (w/v) to 0.1% (w/v),
Indocyanine Green, if present, has a concentration within the range of 0.025% (w/v) to 0.1% (w/v), and,
Trypan Blue has a concentration within the range of 0.01% (w/v) to 0.25% (w/v).

The person skilled in the art will comprehend that the composition according to the invention is not limited to a composition comprising two dyes; further to TB and one of the other dyes listed herein, the composition may comprise a further dye or further dyes either selected from the dyes listed herein or other dyes.

It has also surprisingly been demonstrated that diglycerol (DG) (CAS number 627-82-7) and triglycerol (TG) (CAS number 20411-31-8) conveniently increase the density with a slight increase in viscosity of a composition according to the invention, or another composition suitable for staining an ophthalmic structure without increasing osmotic pressure substantially, whereas agents known and used in the art to increase the density such as sugars, sugar alcohols and polyethylene glycol (PEG) increase the osmotic pressure.

The increase in viscosity is beneficial for the reduction in dilution during the sinking process. DG is not even osmotically active and can be used freely to adjust the density of composition according to the invention, or another composition suitable for staining an ophthalmic structure. In addition, DG does not decrease staining efficiency such as prior art density increasing agents like PEG.

Accordingly, the invention further provides for a composition suitable for staining an ophthalmic structure comprising at least a dye and further comprising diglycerol and/or triglycerol as an agent to increase the density and viscosity of the composition. Preferably, the density of the composition comprising diglycerol and/or triglycerol is increased compared to the identical composition not comprising diglycerol and/or triglycerol, is increased by about 1%, 2%, 3%, 4%, 5%, 6%, 7%, 8%, 9%, or preferably by at least about 10%.

In addition, the invention further provides for a composition according to the invention, further comprising diglycerol and/or triglycerol as an agent to increase the density and viscosity of the composition. A preferred composition according to the invention comprises Trypan Blue and Methyl Blue, Trypan Blue and Methylene Blue or Trypan Blue and Janus Green. Preferably, in such compositions, the total concentration of the diglycerol and/or triglycerol is within the range of about 0.1% to about 25% (v/v), preferably within the range of about 2% to about 20% (v/v), more preferably within the range of about 3% to about 6% (v/v). More preferably, in such composition, the total concentration of the diglycerol and/or triglycerol is within the range of 0.1% to 25% (v/v), preferably within the range of 2% to 20% (v/v), more preferably within the range of 3% to 6% (v/v).

The physiologically normal osmotic pressure is around 300 mosmol/kg, and deviations above about +50 mosmol/kg from the normal value may eventually lead to cellular damage. Accordingly, the osmotic pressure of the composition according to the invention is preferably within the range of about 250 mosmol/kg to about 350 mosmol/kg, more preferably from about 275 mosmol/kg to about 325 mosmol/kg, more preferably from about 285 mosmol/kg to about 315 mosmol/kg, more preferably from about 290 mosmol/kg to about 310 mosmol/kg, more preferably from about 295 mosmol/kg to about 305 mosmol/kg and most preferably is about 300 mosmol/kg. More preferably, the osmotic pressure of the composition according to the invention is preferably within the range of 250 mosmol/kg to 350 mosmol/kg, more preferably from 275 mosmol/kg to 325 mosmol/kg, more preferably from 285 mosmol/kg to 315 mosmol/kg, more preferably from 290 mosmol/kg to 310 mosmol/kg, more preferably from 295 mosmol/kg to 305 mosmol/kg and most preferably is 300 mosmol/kg.

It has also surprisingly been demonstrated that gadolinium complexes such as those used in magnetic resonance imaging (MRI) substantially enhance the staining of dyes in ophthalmic staining from barely discernable to evident and strong. This phenomenon allows for a reduction in dye concentration which is associated with a reduction in risk for the patient. In addition, it was demonstrated that gadolinium complexes such as those used in MRI increase the density of composition suitable for staining an ophthalmic structure such that excellent sinking is observed. For the same increase in density, the gadolinium complexes contribute less to osmotic pressure than the sugars, sugar alcohols and PEG that are routinely used in the art.

Accordingly, the invention further provides for a composition suitable for staining an ophthalmic structure comprising at least a dye and further comprising a Gadolinium complex to enhance the density and to enhance staining of the ophthalmic structure. In addition, the invention further provides for a composition according to the invention, further comprising a Gadolinium complex to enhance the density and to enhance staining of the ophthalmic structure. A preferred composition according to the invention comprises Trypan Blue and Methyl Blue, Trypan Blue and Methylene Blue or Trypan Blue and Janus Green. Preferably, the density of the composition comprising a Gadolinium complex is increased compared to the identical composition not comprising a Gadolinium complex is increased by about 1%, 2%, 3%, 4%, 5%, 6%, 7%, 8%, 9%, or preferably by at least about 10%. Preferably, in such compositions, the concentration of the Gadolinium complex is within the range of about 0.01 M to about 0.1 M, preferably within the range of about 0.01 M to about 0.05 M, more preferably in the range of about 0.02 M to about 0.04 M.

More preferably, in such composition, the concentration of the Gadolinium complex is within the range of 0.01 M to 0.1 M, preferably within the range of 0.01 M to 0.05 M, more preferably in the range of 0.02 M to 0.04 M.

Preferably, the gadolinium complex is a complex with limited or no toxicity such as gadopentetate (CAS number 80529-93-7, gadoterate (CAS number 72573-82-1, gadodiamide (CAS number 122795-43-1, gadoteridol (CAS number 120066-54-8, gadoversetamide (CAS number 131069-91-5, gadobenate (CAS number 113662-23-0, gadobutrol (CAS number 138071-82-6, gadoxetate (CAS number 135326-11-3, gadofosveset (CAS number 193901-90-5, preferably gadopentetate, gadobutrol and gadoterate, more preferably gadopentetate.

The compositions according to the various aspects of the invention can conveniently be provided in a kit.

Accordingly, the invention provides for a kit of parts comprising a composition according to the invention, wherein the kit comprises the compounds within a single composition or comprises the compounds in multiple compositions. The kit may further comprise containers, instructions for use and the like.

The compositions according to the invention can, as described earlier herein, conveniently be used, preferably as a surgical adjuvant, in ophthalmic surgeries defined previously herein.

Accordingly, provided is a method of treatment comprising ophthalmic surgery comprising staining of an ophthalmic structure using a composition or kit according to the invention.

In addition, there is provided the use of a composition or kit according to the invention for the preparation of a medicament, preferably a medicament for ophthalmic surgery, said surgery preferably comprising staining of an ophthalmic structure.

In addition, there is provided the use of a composition or kit according to the invention for use as a medicament, preferably for use in a method for ophthalmic surgery, preferably comprising staining of an ophthalmic structure.

In addition, there is provided a method for staining an ophthalmic structure, preferably an ophthalmic membrane using a composition or kit according to the invention.

Herein, the ophthalmic structure is preferably an ophthalmic membrane, more preferably the internal limiting membrane (ILM) and/or the epiretinal membrane (ERM) and/or the anterior cavity. Other preferred ophthalmic structures and surgeries are selected from the group consisting of the anterior capsule, the anterior segment, the deep lamellar keratoplasty, keratoplasty, Descemet's membranes, cataract surgery, conjunctival surgery and the anterior vitreous.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less about 10% of the value (a range from 9 to 11).

All patent and literature references cited in the present specification are hereby incorporated by reference in their entirety.

### FIGURE LEGEND

FIGS. 1A-1J. Retinas stained with various dye compositions (see examples for further details).

FIG. 1A: 0.1% TB and 5% DG

FIG. 1B: 0.1% TB, 0.025% CV and 5% DG

FIG. 1C: 0.1% TB, 0.025% MG and 5% DG

FIG. 1D: 0.1% TB, 0.025% MV and 5% DG

FIG. 1E: 0.1% TB, 0.025% EV and 5% DG

FIG. 1F: 0.1% TB, 0.025% EB and 5% DG

FIG. 1G: 0.1% TB, 0.025% JG and 5% DG

FIG. 1H: 0.1% TB and 0.025 M gadopentetate

FIG. 1I: 0.1% TB and 0.05 M gadobutrol

FIG. 1J: 0.1% TB and 0.025 M gadoterate

### EXAMPLES

The present invention is further described by the following examples which should not be construed as limiting the scope of the invention.

### Preparation of Stain Solutions

As an example of the preparation of a dye solution, in one exemplary composition, 10 mg TB and 2.5 mg MG are dissolved in 9.5 mL of a physiological buffer solution (consisting of 0.15 M NaCl and 10 mM NaH₂PO₄, adjusted with a NaOH solution to pH=7.2, phosphate-buffered saline, PBS), then adding 500 mg diglycerol. In another exemplary composition, 10 mg TB are dissolved in 8.5 mL of a physiological buffer solution (consisting of 0.15 M NaCl and 10 mM NaH₂PO₄, adjusted with a NaOH solution to pH=7.2), then adding 1 mL of a solution of a solution of 7.5 mg JG in the above-mentioned buffer solution, and 500 μL of a 0.5 M solution of gadobutrol in distilled water.

Other exemplary compositions are prepared in a similar manner.

Staining with Dye Preparations

As an exemplary procedure for demonstrating the intensity of staining, porcine eyes obtained from a local slaughterhouse were used within the first 6 hours after the enucleation. The eyes were kept on ice until used.

The upper part of the eye, including the cornea, iris, and lens, was removed with an orbital section. The vitreous body was removed to get access to the retina. The retina was rinsed three times with phosphate-buffered saline (PBS). Dye solutions were applied to the retina for 30 seconds, and the retina was then washed three times with PBS.

### Photographic Recording of the Result of the Staining

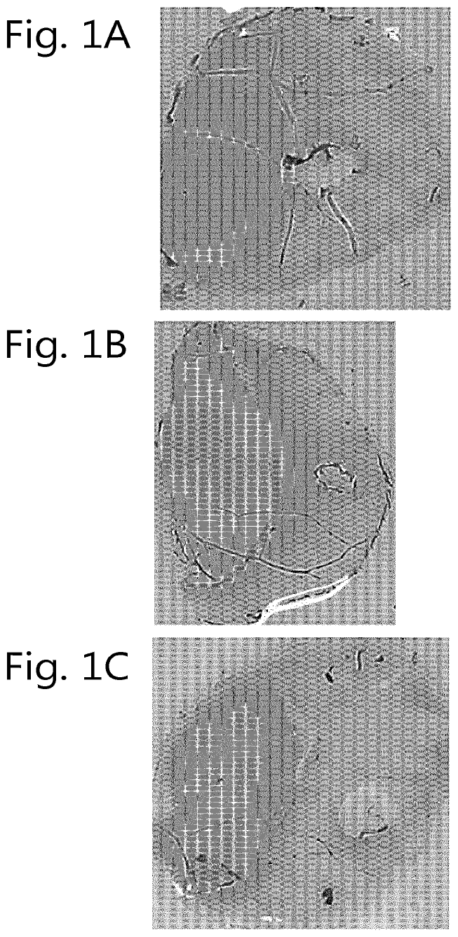

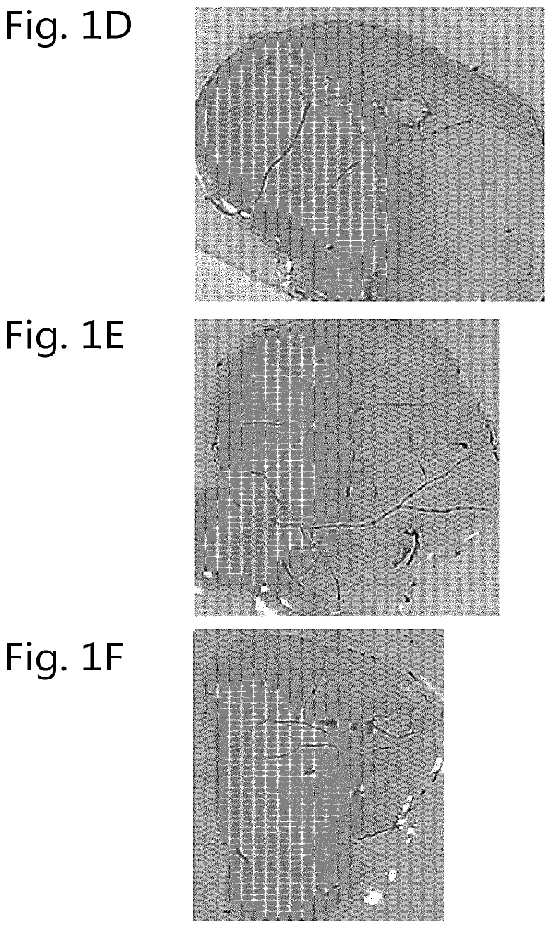

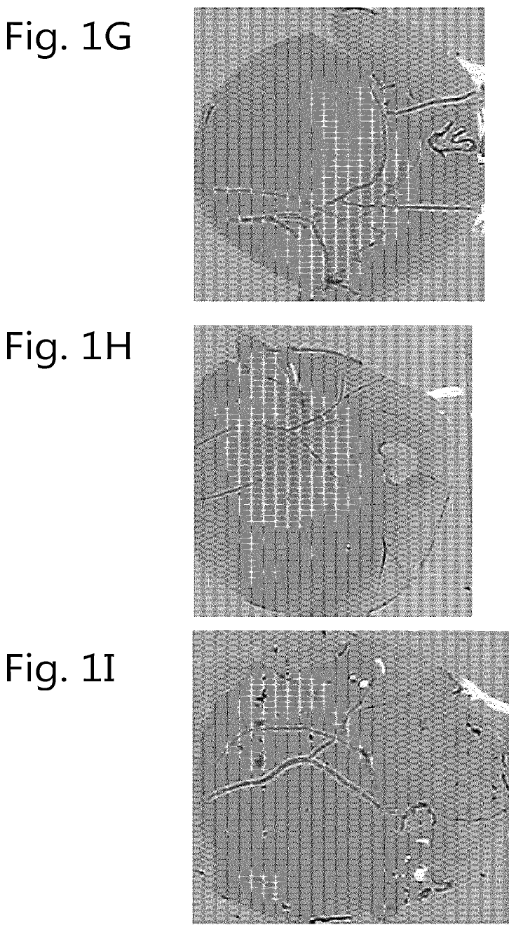

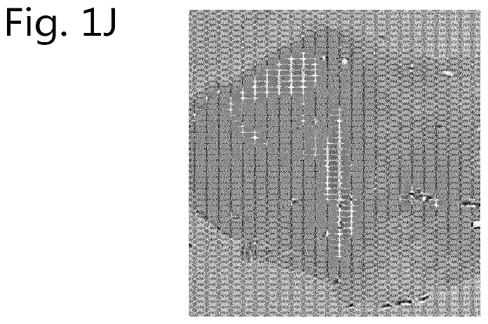

As an example of the visual impression of the stained retina, photos were taken. For photographic recording of the result of the staining, the staining was limited by applying a glass tube of a diameter of 10 mm. Care was taken that no dye could reach the cellular layer of the retina. After the washing and staining, a 10-mm circular section of the retina, including part of the stained area and part of the unstained area, was removed, mounted on a cover slip, and photographed. FIGS. 1A-J show exemplary staining results of both the state of the art and the staining according to the present invention. In all pictures, the stained area is oriented to the left. All solutions contained 5% DG, so that the clinical situation is simulated, which requires an agent which increases density.

To the person skilled in the art, the improvement of the staining according to the invention is evident.

### Quantification of the Staining Result

As an example of quantification, the pictures in FIGS. 1A-J were quantified by measuring the optical densities (expressed as grey values on a scale from 0 (black) to 255 (white)) of a rectangular area within the stained portion of the retina and a rectangular area outside the stained portion as background (ensuring that no blood vessels where present in the rectangular areas), and calculating the ratio of the grey values of the background to that of the stain. With this method, higher numbers mean higher contrast. Toxicity was measured in ARPE cells. Cells were exposed to the dye mixture for 15 minutes, and then washed with three times with PBS. Then, the cells were incubated with the WST-1 reagent (which measures the mitochondrial dehydrogenase activity present in living cells, but absent in dead cells) and incubated at 37° C. as described before [6]. Toxicity is expressed as percent reduction of cell viability in comparison to a control incubated with PBS instead of the dye solution. The results are depicted in the table here below. For combinations comprising TB and JG, TB and MB, TB and MG, TB and CV, TB and MV, and TB and EB, there was a significant increase in contrast compared with the prior art combination of TB and BBG.

| | | | percent |
|---|---|---|---|
| | n-fold | n-fold increase | reduction in |
| | increase | in contrast | cell viability |
| 0.1% TB | in contrast | over | after 15 min |
| plus additive | over TB | TB + BBG + DG | exposure |
| none | ≡1 | | 8 |
| 0.025% BBG | 0.1 | | 0 |
| 0.025% BBG 5% DG | 1.4 | ≡1 | 0 |
| 0.025% BBG 4% | 0.7 | 0.5 | 11 |
| PEG4000 | | | |
| 0.025M | 1.0 | 0.7 | 0 |
| gadopentetate | | | |
| 0.025M gadobutrol | 1.2 | 0.9 | 5 |
| 0.025% EV 5% DG | 1.4 | 1.0 | 13 |
| 0.0075% JG 5% DG | 2.6 | 1.9 | 0 |
| 0.025% MB 5% DG | 1.8 | 1.3 | 9 |
| 0.025% MG 5% DG | 2.1 | 1.5 | 53 |
| 0.025% CV 5% DG | 2.6 | 1.9 | 2 |
| 0.025% MV 5% DG | 2.8 | 2.0 | 9 |
| 0.025% EB 5% DG | 2.9 | 2.1 | 0 |
| 0.025% MeB 5% DG | 2.1 | 1.5 | 22 |
| 0.1% ICG 5% DG | 1.5 | 1.1 | 2 |

### Density and Viscosity

Density and viscosity can be changed by the addition of diglycerol or a gadolinium complex or a combination of both. For sinking of the dye solution to the bottom of the eye, density is more important than viscosity; the latter reduces, however, possible mixing for solutions which sink slowly. The density of dye solutions can be increased from 1.003 grams per milliliter (at 25° C.) to 1.028, when 10 volume % of a 0.5 M solution of gadopentetate is added to a final concentration of gadopentetate of 0.05 M, and to 1.048 when 20 volume % of a 0.5 M solution of gadopentetate is added to a final concentration of 0.10 M. The resulting viscosities are 1.125 mPa·s and 1.258 mPa·s, respectively. For a 5% addition of named gadopentetate solution, a density of 1.016 gram per milliliter and a viscosity of 1.117 mPa·s is obtained.

For the addition of 5 weight percent of diglycerol, a density of 1.020 grams per milliliter and a viscosity of 1.191 mPa·s is obtained.

These data should be compared to those of a 4 weight percent solution of PEG3350 in PBS, which gives a density of 1.013 gram per milliliter and a viscosity of 1.870 mPa·s. The person skilled in the art will find no difficulty to choose concentrations and combinations of the claimed agents which result in appropriate densities and viscosities. The person skilled in the art will also find no difficulty to adjust the contents of ions of the solutions such that a physiologically compatible osmolarity is achieved.

### REFERENCE LIST

1. Jhanji V, Chan E, Das S, Zhang H, Vajpayee R B (2011) Trypan blue dye for anterior segment surgeries. Eye (Lond) 25:1113-1120
2. Semeraro F, Morescalchi F, Duse S, Gambicorti E, Russo A, Costagliola C (2015) Current Trends about Inner Limiting Membrane Peeling in Surgery for Epiretinal Membranes. J Ophthalmol 2015: Article I D 671905
3. Yamamoto N, Ozaki N, Murakami K (2004) Double Visualization Using Triamcinolone Acetonide and Trypan Blue during Stage 3 Macular Hole Surgery. Ophthalmologica 218:297-305
4. Veckeneer M, Mohr A, Alharthi E, Azad R, Bashshur Z F, Bertelli E, Bejjani R A, Bouassida B, Bourla D, Crespo I C, Fahed C, Fayyad F, Mura M, Nawrocki J, Rivett K, Scharioth G B, Shkvorchenko D O, Szurman P, Van Wijck H, Wong I Y, Wong D S, Frank J, Oellerich S, Bruinsma M, Melles G R (2013) Novel ‘heavy’ dyes for retinal membrane staining during macular surgery: multicenter clinical assessment. Acta Ophthalmol 10.1111/aos.12208
5. Henrich P B, Priglinger S G, Haritoglou C, Schumann R G, Strauss R W, Schneider U, Josifova T, Cattin P C (2013) Quantification of contrast recognizability in sequential epiretinal membrane removal and internal limiting membrane peeling in Trypan Blue-assisted macular surgery. Retina 33:818-824
6. Awad D, Schrader I, Bartok M, Mohr A, Gabel D (2011) Comparative toxicology of trypan blue, brilliant blue G, and their combination together with polyethylene glycol on human pigment epithelial cells. Invest Ophthalmol Vis Sci 52:4085-4090

1. A method of ophthalmic surgery, comprising administration of a composition to a subject, said composition comprising a dye and further comprising diglycerol or triglycerol as an agent to increase the density and viscosity of the composition, wherein the total concentration of the diglycerol or triglycerol in the composition is within the range of 0.1% to 25% (v/v), wherein the composition is sterilized and is for staining an ophthalmic structure during surgery, and wherein the diglycerol or triglycerol increases the staining contrast of the dye

2. The method according to claim 1, wherein the dye in the composition is selected from the group consisting of Trypan Blue, Janus Green, Methyl Green, Methylene Blue, Crystal Violet, Methyl Violet, Ethyl Violet, Evans Blue, Methyl Blue and their respective pharmaceutically acceptable salts and hydrates thereof.

3. The method according to claim 2, wherein the total concentration of the diglycerol and triglycerol in the composition is within the range of 2% to 20% (v/v).

4. The method according to claim 3, wherein the total concentration of the diglycerol and triglycerol in the composition is within the range of 3% to 6% (v/v).

5. The method according to claim 1, wherein the composition further comprises a Gadolinium complex to enhance the density and to enhance staining of the ophthalmic structure.

6. The method according to claim 5, wherein the concentration of the Gadolinium complex in the composition is within the range of 0.01 M to 0.1 M.

7. The method according to claim 1, wherein the composition comprises Trypan Blue or a pharmaceutically acceptable salt or hydrate thereof and further comprises a dye selected from the group consisting of Methyl Blue, Methylene Blue, Janus Green, Methyl Green, Crystal Violet, Methyl Violet, Ethyl Violet, Evans Blue and their respective pharmaceutically acceptable salts and hydrates thereof.

8. The method according to claim 7, wherein the composition comprises Trypan Blue and Methyl Blue, Trypan Blue and Methylene Blue or Trypan Blue and Janus Green.

9. The method according to claim 1, wherein in the composition:
Methyl Blue, if present, has a concentration within the range of 0.001% (w/v) to 0.4% (w/v),
Methylene Blue, if present, has a concentration within the range of 0.001% (w/v) to 0.1% (w/v),
Janus Green, if present, has a concentration within the range of 0.001% (w/v) to 0.1% (w/v),
Methyl Green, if present, has a concentration within the range of 0.001% (w/v) to 0.1% (w/v),
Crystal Violet, if present, has a concentration within the range of 0.001% (w/v) to 0.1% (w/v),
Methyl Violet, if present, has a concentration within the range of 0.001% (w/v) to 0.1% (w/v),
Ethyl Violet, if present, has a concentration within the range of 0.001% (w/v) to 0.1% (w/v),
Evans Blue, if present, has a concentration within the range of 0.001% (w/v) to 0.4% (w/v),
Indocyanine Green, if present, has a concentration within the range of about 0.001% (w/v) to about 0.4% (w/v), and,
Trypan Blue, if present, has a concentration within the range of 0.001% (w/v) to 0.25% (w/v.

10. The method according to claim 9, wherein in the composition:
Methyl Blue, if present, has a concentration within the range of 0.01% (w/v)) to 0.1%,
Methylene Blue, if present, has a concentration within the range of 0.01% (w/v) to 0.1% (w/v),
Janus Green, if present, has a concentration within the range of 0.005% (w/v) to 0.01% (w/v),
Methyl Green, if present, has a concentration within the range of 0.01% (w/v) to 0.05% (w/v),
Crystal Violet, if present, has a concentration within the range of 0.01% (w/v) to 0.05% (w/v),
Methyl Violet, if present, has a concentration within the range of 0.01% (w/v) to 0.05% (w/v),
Ethyl Violet, if present, has a concentration within the range of 0.01% (w/v) to 0.05% (w/v),
Evans Blue, if present, has a concentration within the range of 0.025% (w/v) to 0.1% (w/v),
Indocyanine Green, if present, has a concentration within the range of 0.025% (w/v) to 0.1% (w/v), and,
Trypan Blue, if present, has a concentration within the range of 0.01% (w/v) to 0.25% (w/v).

11. The method according to claim 1, wherein the ophthalmic structure is the internal limiting membrane (ILM) the epiretinal membrane (ERM) or the anterior cavity.
